(19) United States
(12) Patent Application Publication
(10) Pub. No.: US 2025/0377352 A1
(43) Pub. Date: Dec. 11, 2025

(54) DIGITALIZED HUMAN ORGANOIDS AND METHODS OF USING SAME

(71) Applicant: CHILDREN'S HOSPITAL MEDICAL CENTER, Cincinnati, OH (US)

(72) Inventors: Takanori TAKEBE, Cincinnati, OH (US); Masaki Kimura, Cincinnati, OH (US)

(73) Assignee: CHILDREN'S HOSPITAL MEDICAL CENTER, Cincinnati, OH (US)

(21) Appl. No.: 19/259,903

(22) Filed: Jul. 3, 2025

Related U.S. Application Data
(63) Continuation of application No. 16/956,230, filed on Jun. 19, 2020, now Pat. No. 12,379,372.
(60) Provisional application No. 62/608,695, filed on Dec. 21, 2017.

(51) Int. Cl. G01N 33/50; C12N 5/071; C12Q 1/6881; G16H 20/40

(52) U.S. Cl. CPC G01N 33/5088; C12N 5/0671; C12Q 1/6881; G16H 20/40; C12N 2501/12; C12N 2501/727; C12N 2503/04; C12N 2506/45; C12Q 2600/112; C12Q 2600/136; G01N 2333/76; G01N 2405/00

(57) ABSTRACT

Disclosed are digitized organoids comprising a detectable sensor, such as, for example, a Radio frequency identification (RFID) based sensor. Further disclosed are methods for making the digitized organoids. The disclosed methods allow for self-assembly mediated incorporation of ultracompact RFID sensors into organoids. Methods of using the digitized organoids are also disclosed.

### CROSS-REFERENCE TO RELATED APPLICATIONS

This application is a continuation of U.S. application Ser. No. 19/956,230, filed Jun. 19, 2020, which is a national phase application under 35 U.S.C. § 371 of International Application No. PCT/US2018/067057, filed Dec. 21, 2018, which claims priority to and benefit of U.S. Provisional Application Ser. No. 62/608,695, filed Dec. 21, 2017, the contents of which application are incorporated into the present application by reference in their entireties.

### BACKGROUND

In recent years, radio frequency identification (RFID) has been examined in various healthcare contexts. For example, implanting RFID microchips in animals and humans allows for positive identification of specific individuals. An additional interesting medical application of RFID includes incorporation into oral medication known as a “digital pill” with the aim of improving patients' adherence in chronic conditions. Diverse applications of RFID have the potential to provide innovative solutions to various biomedical challenges. There remains a need for advancements in tracking in vitro and in vivo processes, in drug discovery, and in understanding disease mechanisms. Realistic RFID applications in tissue culture context, however, necessitates viable methods to incorporate the microchip into biological tissue without impairing the tissue's native structure and functions. No studies to date have examined RFID applications in tissue culture contexts, presumably due to a lack of viable methods to incorporate the microchip into biological tissue without impairing the tissue's native structure and functions. The instant disclosure seeks to address one or more of the aforementioned needs in the art.

### SUMMARY OF THE INVENTION

Disclosed are digitized organoids comprising a detectable sensor, such as, for example, a Radio frequency identification (RFID) based sensor. Further disclosed are methods for making the digitized organoids. The disclosed methods allow for self-assembly mediated incorporation of ultracompact RFID sensors into organoids. Methods of using the digitized organoids are also disclosed.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1A-1D. Concept of organoid digitalization with O-Chip. (1A) A schematic of organoid digitalization strategy. Integration of O-Chip into organoids makes it possible to digitalize organoids. (1B) The size of the O-Chip is 0.46×0.48 [μm2] and this is one of the smallest microchips in the world. (1C) Self-condensation culture with O-Chip 9. Serial pictures show O-Chip being integrated into organoids formed from iPSC-derivatives. (1D) Each organoid completely encompasses one RFID microchip, where the iPSC-derivatives.

FIG. 2A-2E. O-Chip incorporation into human iPSC liver organoid. (2A) A comparison between RiO and Control LO demonstrating no differences in morphology. (2B, 2C) A comparison between RiO and Control LO demonstrating no functional differences. (2B) Gene expression analysis was performed for the liver specific markers ALB, APP and AAT in RIO and Control LO. (2C) An ALB ELISA was performed on collected supernatant from RiO and Control LO. (2D) A representative image of immunostaining for ALB, HNP4A and DAPI on a RiO. (2E) CLP and Rhodamine123 uptake into RiO. Images were taken 10 minutes after each fluorescein exposure. Rectangle indicates O-Chip.

FIG. 3A-3C. Wireless traceability of RiO in vitro and in vivo. (3A) O-Chip integrated into organoids makes it possible to harvest and pool organoids from different IPS cell lines into one well. (3B) RiO cultured in both 96 well U bottom and 24 well plates can be detected through the bottom of the plate with a chip reader. The organoids from different lines are pooled together in each well of a 24 well plate. (3C) RSSI values are detectable and comparable in RiO cultured in either 24 well or 96 well plates and in medium or PBS. (3D) In vivo experiment to localize O-Chips implanted subcutaneously in mouse. Applicant embedded 3 O-Chips under mouse skin and detected each with a chip reader. Shown in picture 6, Applicant marked 3 points and embedded 3 RFID-organoids. (e) RSSI values are detectable when the O-Chips are embedded subcutaneously, although the values are slightly lower than in vitro.

FIG. 4A-4D. (4A) RiO were generated from 7 different iPSC lines derived that includes two Wolman disease patients. The RiO were then pooled together in one culture, and treated with fatty acid. The amount of accumulated lipids were quantified by fluorescent imaging as a method to screen for steatosis. (4B) EPC number allocation per specific donor before freezing. Green highlighted cell lines were two Wolman disease iPSCs, and the other pink ones were five control iPSCs. (4C) A fluorescent image of pooled RiO after steatosis induction demonstrating lipid accumulation using BODIPY. RiO derived from different donors have varying lipid accumulation. Identification of each individual RiO from different iPSC lines is not possible by visual inspection. (4D) Quantification of individual RiO fluorescence intensity, followed by wireless detection of EPC# in RiO by RFID reader. RiO derived from a Wolman disease patient accumulate the most lipids, relative to 5 other control iPSC lines.

FIG. 5A-5C. Reproducible RiO generation from multiple donor-derived iPSC liver organoids. (Related to FIG. 2). 5A) RiO shows similar morphological and immunological profiles to HLO. 5B) morphology of RiO and control HLO. 5C) immunostaining of RiO and control HLO. Middle panel shows higher magnification of central part of RiO shows ALB and HNF4A expression, although thickness of the tissues weaken the fluorescence intensity measurements.

FIG. 6A-6B. 6A depicts measurement workflow of fat fluorescence intensity in RIO (related to FIG. 2). The fat accumulation capacity of RiO was studied using the fatty acid treatment and lipid dye BODIPY® 493/503 for lipids. FIG. 6B shows GTP intensity.

FIG. 7A-7B. 7A depicts detection of iron accumulation in RiO. (Related to FIG. 2). Iron accumulation capacity was studied using the ammonium sulfate treatment and Fe dye FeRhoNox® 540/575. FIG. 7B depicts RFP intensity measurements.

FIG. 8. Shows wireless detection of paraffin embedded RiO. (Related to FIG. 2.) RiO phenotype is similar to normal iPSC liver organoids.

FIG. 9A-9E. Wireless identification of RiO in vitro and in vivo. Related to FIG. 2. (FIG. 9A, FIG. 9B) Scheme of RFID identification in RiO. Generated RiO were placed into 96 and 24 well plastic plates. The RFID signal was detected by the detection probe from the bottom of the plate. FIG. 9C) Measurement of RSSI value under various media conditions. (FIG. 9D) Measurement of RSSI value in vitro and in vivo. (FIG. 9E) Tracking of O-Chip location after implantation. After embedding the O-Chip under mouse skin, FRID signal was measured with a reader directly over the mouse's skin.

FIG. 10A-10D. Thawing and lipid detection of Cryopreserved RIO. (FIG A) Representative morphology of RiO and control LO before cryopreservation. (FIG B) RiO before cryopreservation. (FIG C) Representative morphology of RiO and control LO after thawing. (FIG D) Live-cell lipid imaging of RiO and control LO after thawing.

FIG. 11. Shows that RiO phenotype is similar to normal iPSC LO. (Related to FIG. 4.)

FIG. 12. Shows the difference in lipid accumulation between healthy iPSCs and monogenic iPSC line (Related to FIG. 4.)

### DETAILED DESCRIPTION OF THE INVENTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multi potent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include primary cells, which are cells taken directly from living tissue (e.g. biopsy material) and established for growth in vitro.

RFID is cost-effective technology for addressing personal identification, traceability and environmental considerations especially in transportation industries, which has permeated all facets of modern life1-3. RFID tags, operating wirelessly, collect energy from a nearby reader's interrogating radio waves. Its high-degree of tolerance to tested solutions, solvents, extreme temperatures, and high or low-pressure conditions4, provides the RFID tags with significant advantages over barcodes. The tags are now integrated into cards, clothing, and possessions, as well as implanted into animals and humans. For example, RFID tags are incorporated into cards and are used to pay for mass transit fares on buses, trains, subways, and to collect tolls on highways in many countries. In 2017, the world RFID market which includes tags, readers, software/services for RFID cards, labels, fobs, and all other form factors was worth US $11.2 billion in 2017 and has an estimated 10% annual growth, resulting in an anticipated value of US $18.68 billion by 20265.

In recent years, there has been considerable interest in extending usage of RFID to the healthcare arena. For example, implanting RFID microchips in animals and humans allows for positive identification of specific individuals. Medical application of RFID now includes an oral “digital pill” for chronic conditions, in efforts to improve patients' adherence6,7. The ingested radiofrequency emitter, once activated by gastric pH, emits a radiofrequency signal which is captured by a relay Hub and transmitted to a smartphone, where it provides ingestion data and deliver interventions in real time. Thus, the diverse applications of RFID provide innovative solutions to various biomedical challenges. Similarly, RFID incorporation into cells or tissues can provide advancements in tracking in vitro and in vivo processes, in drug discovery, and in understanding disease mechanisms. Recent application of micro RFID showed the passive intra-cellular delivery and short-term persistence; however, the use of mouse phagocytic cell line limits its broader application8. Thus, realistic RFID applications in tissue culture context necessitates viable methods to incorporate the microchip into biological tissue without impairing the tissue's native structure and functions.

Recently, human organoids have received international attention as an in vitro culture system where human stem cells self-organize into three-dimensional (3D) structures reminiscent of human organs9-11. Organoids, due to their higher phenotypic fidelity to human disease12, are expected to provide a mechanistic assay platform with future potential for drug screening and personalized medicine. It becomes feasible to study human pathological variations by comparing genotypes with phenotype spectrum diseases that includes cystic fibrosis13, steatohepatitis (Ouchi et al., In Revision) and cholestatic disease (Shinozawa et al., In Revision) using a human induced pluripotent stem cell (iPSC) or adult stem cell library. One key feature of organoids is the development of a polarized structure surrounded by basement membrane through a self-assembly process, resulting in a cavitated structure from an aggregated tissue.

Disclosed herein are methods for self-assembly mediated incorporation of ultracompact Radio Frequency Identification (RFID) chips into organoids formed from human pluripotent stem cells. RFID microchip incorporated organoids (“RiO”) emit unique digital signals through the wireless readers, which can be used to decode donor information and localize RiO in vitro. RiO can be stored by freezing, possess their original structure and function, and maintained in long term culture. By taking advantage of donor identifiable organoids, digitalized RiO pools from different donors can be used to conduct a phenotypic screen, followed by detection of specific donors. For example, proof-of-principle experiments successfully identified that the severely steatotic phenotype is caused by a genetic disorder of steatohepatitis. The disclosed methods and compositions allow for a ‘forward cellomics’ approach as a potential strategy to determine the personalized phenotypes in a pooled condition, which is a scalable approach of studying genetic impacts on phenotype as an analogical strategy to forward genetics. Given that the miniaturization of digital devices including biosensors, robotics and cameras is rapidly evolving, bio-integration of microchip is likely to be a powerful approach to expand organoid based medical applications towards drug development, precision medicine, and transplantation. The disclosed methods and compositions may have a wide audience in the fields of bioengineering, biotechnology, material science, computational biology, stem cell biology, regenerative biology, tissue engineering, transplantation, ethics and regulatory science, providing a strategic framework to promote organoid potential for testing human variations.

In one aspect, a digitized organoid is disclosed. The digitized organoid may comprise a detectable sensor, for example, an RFID based sensor, wherein the digitized organoid may comprise a lumen. The detectable sensor may be located within the lumen. The detectable sensor may have a diameter of less than about 1 mm2 in certain aspects.

In one aspect, a pooled organoid composition comprising a plurality of digitized organoids comprising a detectable sensor is disclosed. The plurality of digitized organoids may comprise at least one organoid derived from a first donor and at least one organoid derived from a second donor. In certain aspects, the plurality of organoids may be derived from more than two donors, or more than three donors, or more than four donors, or more than five donors, or more than six donors, or more than seven donors, or more than eight donors, or more than nine donors, or more than ten donors, or up to 100,000 donors, or up to 500,000 donors, or up to one million or two million donors.

The detectable sensor may take a variety of different forms, as will be appreciated by one of ordinary skill in the art. In certain aspects, for example, the detectable sensor may be hydrogel-based, electronics-based, printed electronics-based, magnetic-based, micro-robotic based, or a combination thereof. Suitable detectable sensors will be readily appreciated by one of ordinary skill in the art and it should be appreciated that the abovementioned list is non-limiting.

In one aspect, the detectable sensor may be configured to detect and measure one or more biological parameters, including physiological, chemical, or electrical parameters. Exemplary parameters include pH, temperature, pressure, stiffness, elasticity, viscosity, osmotic pressure, DNA, mRNA, protein, carbohydrate (glucose), oxygen, metabolites (ammonia, urea, lactate,) reactive oxygen species (ROS), or combinations thereof.

In one aspect, the organoid may comprise an identifier unique to the donor that is the source of the cells which have been developed into an organoid. The identifier may be encoded in the detectable sensor. Detection of the unique identifier allows for correlation of the RFID to the individual donor, and further, the measured parameter with the individual donor. This data may then be pooled and collected and optionally analyzed to determine associations related to the donor population and the detected/measured parameter.

In one aspect, the digitized organoid or plurality of digitized organoids may comprise an organoid selected from a liver organoid, a small intestinal organoid, a large intestinal organoid, a stomach organoid, and combinations thereof. In certain aspects, the organoid is a human liver organoid, and may include a liver organoid having a steatotic phenotype.

In one aspect, a method of making an RFID-integrated organoid (RiO or “digitized organoid”) is disclosed. The method may comprise the step of contacting a plurality of definitive endoderm cells with a micro-RFID chip in a differentiation medium for a period of time sufficient to allow co-aggregation of definitive endoderm cells and the micro-RFID chip, wherein the contacting step may be carried out in the presence of at least 2% v/v basement membrane matrix; wherein the co-aggregation may form an organoid comprising a lumen; wherein the definitive endoderm cells may be provided as single cells derived from a spheroid; wherein the spheroid may be derived from a definitive endoderm derived from a precursor cell; and wherein the micro-RFID chip may be localized in the lumen.

In one aspect, the spheroid may be derived from a precursor cell, for example, an iPSC. The precursor cell may be contacted with Activan A and BMP4 for a period of time sufficient to form definitive endoderm. The definitive endoderm may then be incubated with a culture favorable to forming a spheroid which is further capable of being developed into an organoid of interest.

In one aspect, the organoid of interest may be esophageal. In this aspect, the definitive endoderm may be contacted with an FGF signaling pathway activator, for example, a small molecule or protein that activates the FGF signaling pathway, for example, FGF4, and a BMP inhibitor for a period of time sufficient to form a spheroid capable of developing into an esophageal organoid. Suitable BMP activators will be appreciated by one of ordinary skill in the art and may include small molecules and/or proteins that activate the pathway. Combinations of any of the foregoing may be used.

In one aspect, the organoid of interest may be stomach. In this aspect, the definitive endoderm may be contacted with retinoic acid, a Wnt signaling pathway activator (for example CHIR99021, but which may further include any small molecule pathway activator or protein that activates the pathway such as a GSK3 inhibitor) and optionally Activin for a period of time sufficient to form a spheroid capable of developing into a fundal organoid.

In one aspect, the organoid of interest may be stomach. In this aspect, the definitive endoderm may be contacted with retinoic acid, Wnt signaling pathway activator (for example CHIR99021, but which may further include any small molecule pathway activator or protein that activates the pathway such as a GSK3 inhibitor), Activin, a MEK activator, and a BMP activator, for a period of time sufficient to form a spheroid capable of developing into an acid secreting stomach organoid. Suitable BMP activators will be appreciated by one of ordinary skill in the art and may include small molecules and/or proteins that activate the pathway. Combinations of any of the foregoing may be used.

In one aspect, the organoid of interest may be small intestine. In this aspect, the definitive endoderm may be contacted with an FGF signaling pathway activator, for example, a small molecule or protein that activates the FGF signaling pathway, for example, FGF4, and a Wnt signaling pathway activator (for example CHIR99021, but which may further include any small molecule pathway activator or protein that activates the pathway such as a GSK3 inhibitor) for a period of time sufficient to form a spheroid capable of developing into a small intestinal organoid. Combinations of any of the foregoing may be used.

In one aspect, the organoid of interest may be colon. In this aspect, the definitive endoderm may be contacted with an FGF signaling pathway activator, for example, a small molecule or protein that activates the FGF signaling pathway, for example, FGF4, a Wnt signaling pathway activator (for example CHIR99021, but which may further include any small molecule pathway activator or protein that activates the pathway such as a GSK3 inhibitor), and a BMP activator, for a period of time sufficient to form a colonic organoid. Suitable BMP activators will be appreciated by one of ordinary skill in the art and may include small molecules and/or proteins that activate the pathway. Combinations of any of the foregoing may be used.

In one aspect, the organoid of interest may be hepatic. In this aspect, the definitive endoderm may be contacted with an FGF signaling pathway activator, for example, a small molecule or protein that activates the FGF signaling pathway, for example, FGF4, a BMP activator, and/or retinoic and for a period of time sufficient to form a spheroid capable of developing into a hepatic organoid.

In one aspect, the organoid of interest may be liver. In this aspect, the definitive endoderm may be contacted with an FGF signaling pathway activator for a period of time sufficient to form a spheroid capable of developing into a liver organoid.

In one aspect, the organoid of interest may be bile duct. In this aspect, the definitive endoderm may be contacted with an FGF signaling pathway activator, for example, a small molecule or protein that activates the FGF signaling pathway, for example, FGF10, a EGF signaling pathway activator, Activin and retinoic acid, for a period of time sufficient to form a biliary organoid.

In one aspect, the organoid of interest may be pancreas. In this aspect, the definitive endoderm may be contacted with retinoic acid (RA), an AMPK inhibitor (dorsomorphin), a TGF inhibitor (SB-431542), an FGF signaling pathway activator, for example, a small molecule or protein that activates the FGF signaling pathway, for example, FGF4, and KAAD-cyclopamine, for a period of time sufficient to form a pancreatic organoid.

In one aspect, the organoid of interest may be lung. In this aspect, the definitive endoderm may be contacted with an FGF signaling pathway activator, for example, a small molecule or protein that activates the FGF signaling pathway, for example, FGF10 and FGF7, a Wnt signaling pathway activator (for example CHIR99021, but which may further include any small molecule pathway activator or protein that activates the pathway such as a GSK3 inhibitor), and a BMP (such as BMP4) and a retinoic acid, for a period of time sufficient to form a lung organoid. Suitable BMP activators will be appreciated by one of ordinary skill in the art and may include small molecules and/or proteins that activate the pathway. Combinations of any of the foregoing may be used.

In one aspect, the organoid of interest may be kidney. In this aspect, the definitive endoderm may be contacted with a retinoic acid, a FGF signaling pathway activator, for example, a small molecule or protein that activates the FGF signaling pathway, for example, FGF4, a Wnt signaling pathway activator (for example CHIR99021, but which may further include any small molecule pathway activator or protein that activates the pathway), GDNF, for a period of time sufficient to form a kidney organoid.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. Suitable FGF pathway activators will be readily understood by one of ordinary skill in the art. Exemplary FGF pathway activators include, but are not limited to: one or more molecules selected from the group consisting of FGF1, FGF2, FGF3, FGF4, FGF10, FGF11, FGF12, FGF13, FGF14, FGF15, FGF16, FGF17, FGF18, FGF19, FGF20, FGF21, FGF22, and FGF23. In some embodiments, siRNA and/or shRNA targeting cellular constituents associated with the FGF signaling pathway may be used to activate these pathways.

In some embodiments, DE culture is treated with the one or more molecules of the FGF signaling pathway described herein at a concentration of 10 ng/ml or higher; 20 ng/ml or higher; 50 ng/ml or higher; 75 ng/ml or higher; 100 ng/ml or higher; 120 ng/ml or higher; 150 ng/ml or higher; 200 ng/ml or higher; 500 ng/ml or higher; 1,000 ng/ml or higher; 1,200 ng/ml or higher; 1,500 ng/ml or higher; 2,000 ng/ml or higher; 5,000 ng/ml or higher; 7,000 ng/ml or higher; 10,000 ng/ml or higher; or 15,000 ng/ml or higher. In some embodiments, concentration of signaling molecule is maintained at a constant throughout the treatment. In other embodiments, concentration of the molecules of a signaling pathway is varied during the course of the treatment. In some embodiments, a signaling molecule in accordance with the present invention is suspended in media comprising DMEM and fetal bovine serine (PBS). The PBS can be at a concentration of 2% and more; 5% and more; 10% or more; 15% or more; 20% or more; 30% or more; or 50% or more. One of skill in the art would understand that the regiment described herein is applicable to any known molecules of the signaling pathways described herein, alone or in combination, including but not limited to any molecules in the FGF signaling pathway.

Suitable FGF signaling pathway activators will be readily appreciated by one of skill in the art. In one aspect, the FGF signaling pathway activator may be selected from a small molecule or protein FGF signaling pathway activator, FGF1, FGF2, FGF3, FGF4, FGF10, FGF11, FGF12, FGF13, FGF14, FGF15, FGF16, FGF17, FGF18, FGF19, FGF20, FGF21, FGF22, FGF23, or combinations thereof. The WNT signaling pathway activator may be selected from a small molecule or protein Wnt signaling pathway activator such as Lithium Chloride; 2-amino-4,6-disubstituted pyrimidine (hetero) arylpyrimidines; IQ1; QS11; NSC668036; DCA beta-catenin; 2-amino-4-[3,4-(methylenedioxy)-benzylamino]-6-(3-methoxyphenyl) pyrimidine, Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, a GSK3 inhibitor, preferably CHIRON, or combinations thereof. In one aspect, the BMP activator may be selected from BMP2, BMP4, BMP7, BMP9, small molecules that activates the BMP pathway, proteins that activate the BMP pathway, and may include the following: Noggin, Dorsomorphin, LDN189, DMH-1, ventromophins, and combinations thereof. Suitable GSK3 inhibitors will be readily understood by one of ordinary skill in the art. Exemplary GSK3 inhibitors include, but are not limited to: Chiron/CHIR99021, for example, which inhibits GSK3p. One of ordinary skill in the art will recognize GSK3 inhibitors suitable for carrying out the disclosed methods. The GSK3 inhibitor may be administered in an amount of from about 1 μM to about 100 μM, or from about 2 μM to about 50 μM, or from about 3 μM to about 25 μM. One of ordinary skill in the art will readily appreciate the appropriate amount and duration. In some aspects, siRNA and/or shRNA targeting cellular constituents associated with the Wnt and/or FGF signaling pathways may be used to activate these pathways. In one aspect, a differentiation medium comprising Rho-associated kinase (ROCK) inhibitor or Y-27632 may be used.

In one aspect, any organoid of the instant disclosure may be characterized by having a cavitated structure comprising a polarized structure surrounded by a basement membrane. In one aspect, the RiO may comprise native structures such as biliary canaliculi, microvilli.

In one aspect, the RiO may comprise precursor cells from more than one individual. In one aspect, the more than one individual donor may each have been diagnosed with a disease of interest. In one aspect, the more than one individual donor may be characterized by having a pre-determined disease state.

In one aspect, a method of screening a cell population based on phenotype is disclosed. The method may comprise the step of detecting at least two features of an organoid in a pooled organoid composition, wherein the first feature may be the identity of the donor individual, and wherein the second feature may be a cell phenotype. Non-limiting examples of a cell phenotype may be, for example, cell viability, pathological cell morphology, cell survival, or combinations thereof. In one aspect, the first feature may be identified using a microchip located in a lumen of an organoid, and the second feature may be identified using a second detection method, for example, cell fluorescence or radioactivity. The donor may be assayed for a genotype, and the genotype may be correlated with the second feature. In one aspect, the second feature may be drug toxicity or efficacy, wherein the method may further comprise the step of contacting the pooled organoid composition with a drug of interest.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Radio frequency identification (RFID) is a cost-effective and durable method to trace and track individual objects in multiple contexts by wirelessly providing digital signals, and is widely used in manufacturing, logistics, retail markets, transportation, and most recently human implants. Here, Applicant has successfully applied the digitalization principles to biological tissues by developing an ultra-compact RFID chip incorporated organoid (RiO). Demonstrated herein is a 0.4 mm RFID chip integrated inside the self-assembling organoids from 10 different induced pluripotent stem cell (iPSC) lines from healthy and diseased donors. Applicant was able to decode RiO' s specific donors and localize RiO in vitro by a wireless reader/writer. RiOs can be maintained for a minimum of 20 days in culture, can be frozen/thawed through a standard preservation method, and exhibit essential hepatic functions similar to control human liver organoids. Furthermore, Applicant demonstrated that the digitalized RiO from different donors could be used to conduct a phenotypic screen on a pool of RiO, followed by detection of each specific donor in situ. Applicant's proof-of-principle experiments demonstrated that the severely steatotic phenotype could be identified by RFID chip reading and was specific to a genetic disorder of steatohepatitis. Given evolving advancements surrounding RFID technology including miniaturization, environmental sensing, and data-writing capabilities, the digitalization principle outlined here will greatly expand the organoid medicine potential towards drug development, precision medicine and transplant applications.

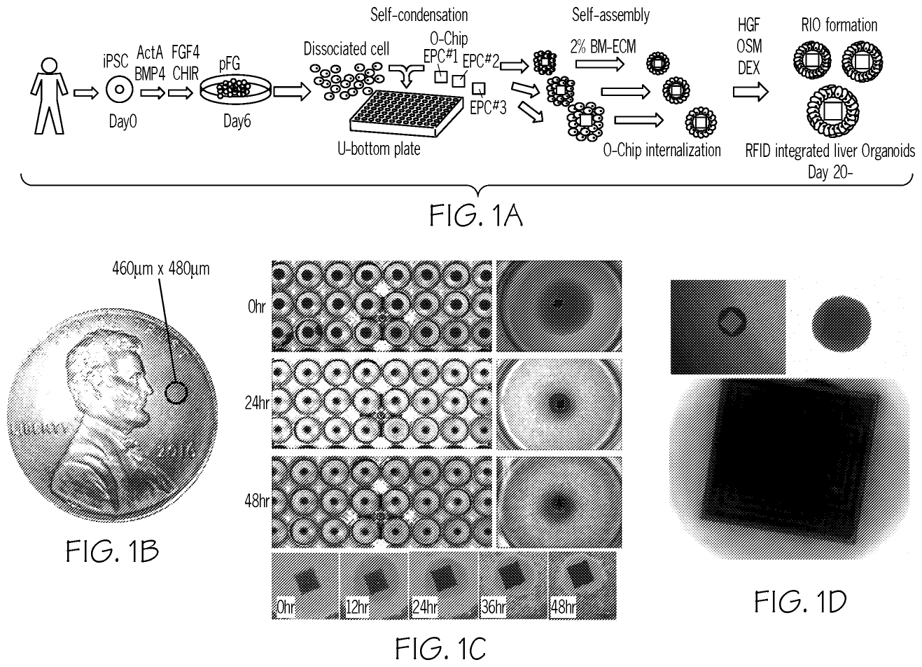

Applicant hypothesized that aggregation-mediated self-assembling process would enable the successful internalization of miniature chips into biological tissues without compromising the native functions of the tissues. Ultracompact RFID-chips were integrated into re-aggregated iPSC-derived endoderm spheroids prior to self-assembly. Recent advancements in miniaturization have generated ultra-compact RFID microchips ranging in size from 10 μm to 600 μm7,8,14. For simplicity and accessibility, commercially available organoid-scale RFID chips were used, herein defined as an “O-Chip.” FIG. 1A depicts an overall strategy. Each O-Chip is 460×480 μm, and has a S12-bit memory area (FIG. 1B). By applying a specific wavelength into a coiled antenna, each O-Chip receives data sent from the reader/writer, and with energy driven by this electric current the O-Chip wirelessly sends information stored in its memory. O-Chip operates wirelessly from readers across distances of up to about 1-2 mm.

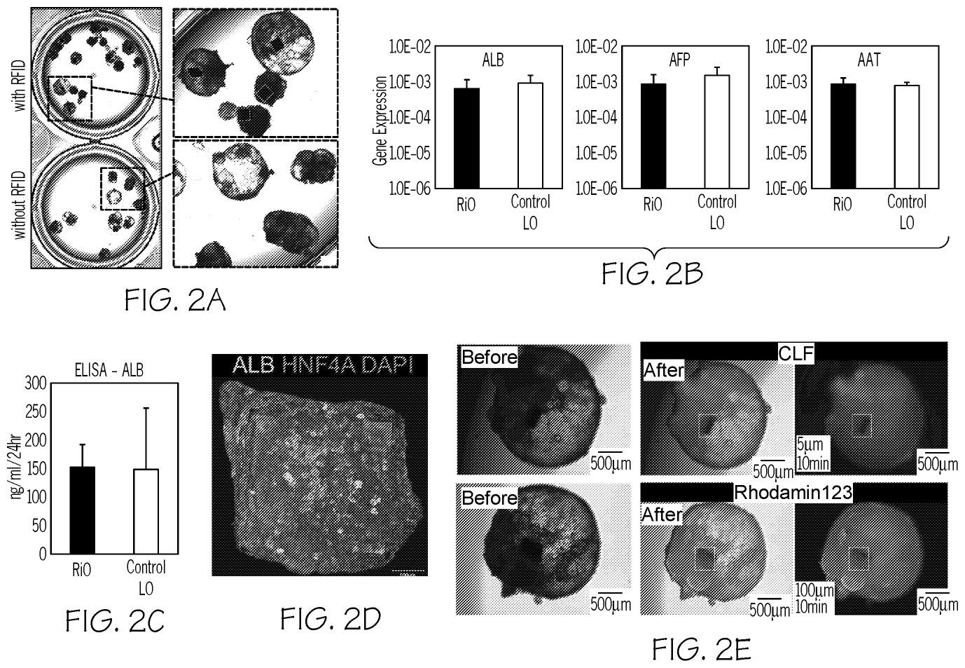

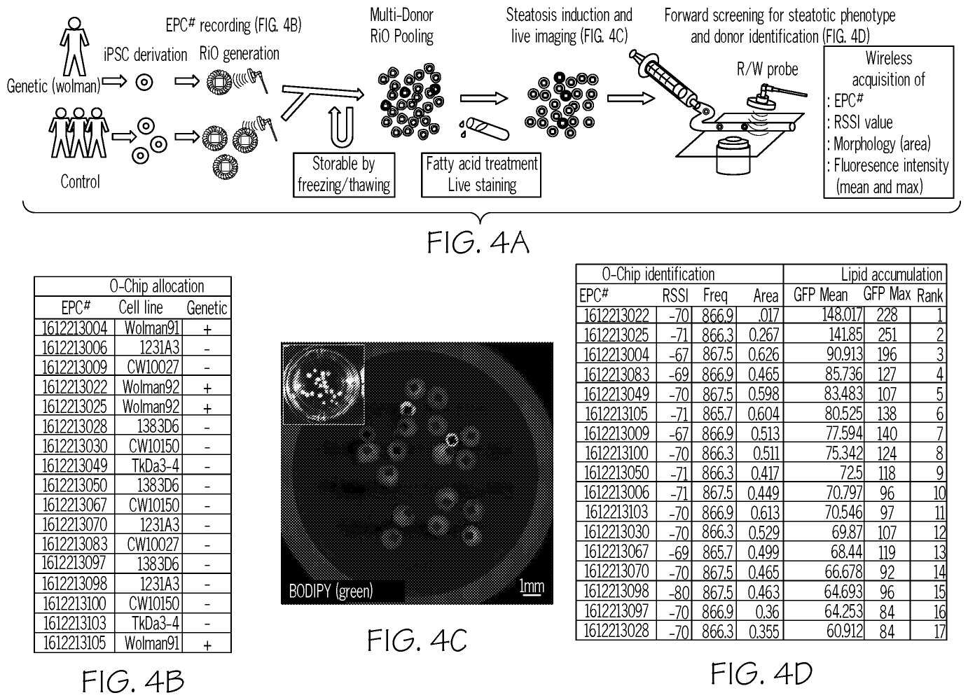

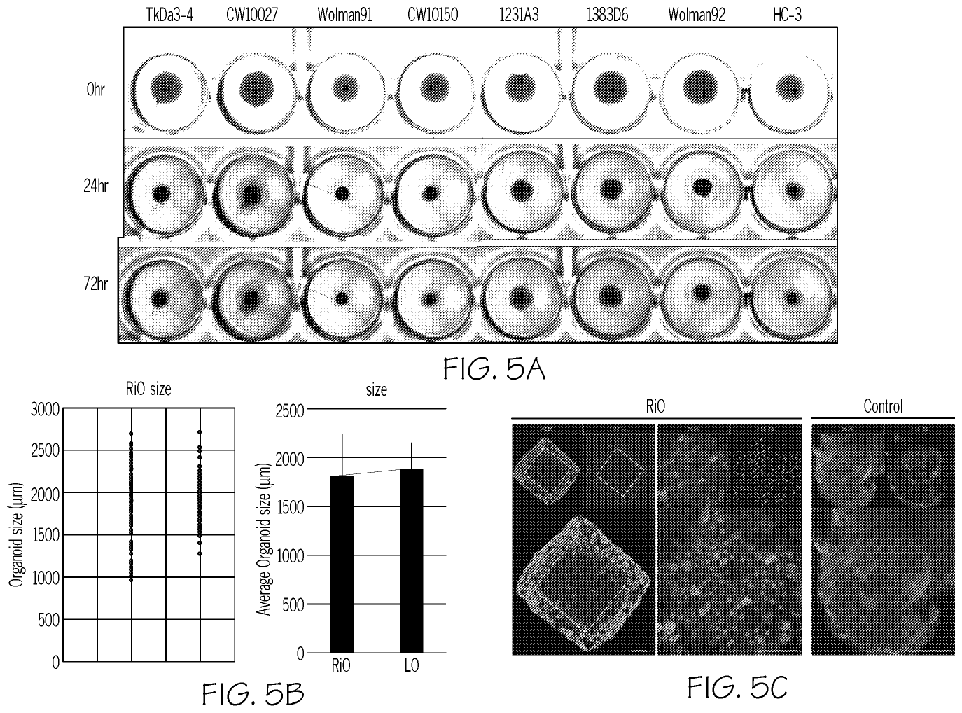

To test O-Chip integration into biological tissues, protocols were modified for generating human liver organoids derived from induced pluripotent stem cells (iPSC) (Shinozawa et al, In Revision). Specifically, human iPSC were initially differentiated into posterior foregut organoids by sequential Activin and FGF4/CHIR exposure. At day 6, foregut organoids were dissociated and seeded into 96 well plates, followed by O-Chip plating. Once re-aggregated tissues were formed, tissues were treated with a 2% laminin-rich basement membrane matrix to induce self-assembly or polarization. After 3 days in culture RiO (FIG. 1C, 1D) covered by hepatic epithelial cells were successfully generated (FIG. 2A). Applicant confirmed reproducible RiO generation by using eight different donor-derived iPSC lines (FIG. 4, FIGS. 5A and 5B). The RiO formation efficiency, which means incorporation of RFID into the organoid, was very high and was 95% or more (92 of 96 organogens succeeded). Collectively, O-Chip can be efficiently integrated inside iPSC-derived organoids.

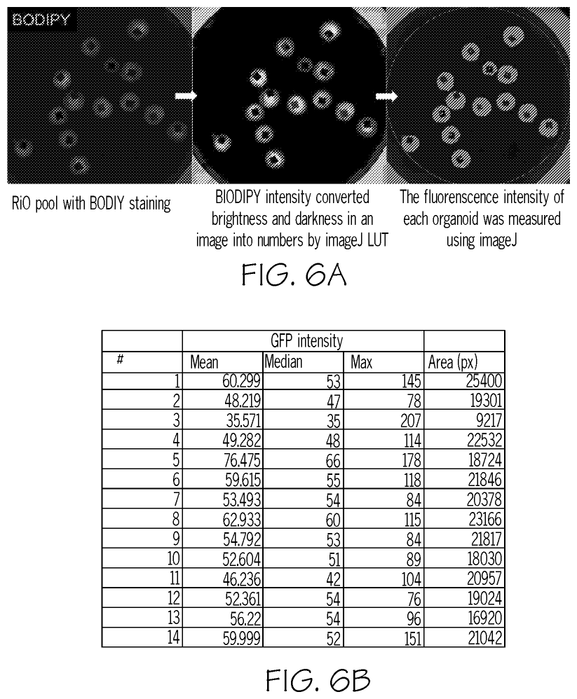

To compare the RiO with standard liver organoids (Control LO), a comparison of their morphology was first performed, and no morphological abnormalities in RiO were observed compared with Control LO (FIG. 2A). Next, Applicant performed a quantitative polymerase chain reaction (qPCR) analysis to detect liver specific genes (FIG. 2B). qPCR analysis revealed that RiO expressed equivalent levels of albumin (ALB), alpha-fetoprotein (APP) and alpha-1-antitrypsin (AAT) compared to Control LO (FIG. 2B). To confirm these findings, Applicant performed an enzyme linked immunosorbent assay (ELISA) of culture supernatant for the liver specific protein ALB, and found comparable protein levels between the RiO and Control LO (FIG. 2C). Applicant found that the RiO secreted ALB at levels of 152.9 ng/ml after 24 hr, which is very similar to Control LO (1.02-fold) (FIG. 2C). Immunofluorescent whole mount staining confirmed the presence of ALB+ cells inside the RiO (FIG. 2D). Another liver marker, HNF4a, was found in the nucleus of ALB+ cells (FIG. 2D and FIG. 6C).

To further investigate the functional capacity of RiO, Applicant analyzed bile transport potential. The bile transport capacity of RiO was studied using the fluorescent bile acids cholyl-lysyl-fluorescein (CLP) and rhodamine 123, which are the substrates for the Bile Salt Efflux Pump (BSEP) and the cholangiocyte surface glycoprotein multidrug resistance protein-1 (MDR1), respectively. Applicant has previously shown that Control LO have the capacity to uptake CLP and rhodamine 123 inside the organoids through BSEP and MDR1. Consistent with this, CLP and rhodamine 123 were absorbed into the cells of RiO within minutes of the addition of the fluorescent bile acids. The fluorescent acids were then excreted and accumulated in the lumen of RiO (FIG. 2E). The fat accumulation capacity of RiO was studied using the fatty acid treatment and lipid dye BODIPY 493/503 for lipids. We treated the RiO with fatty acids and then visualized the amount of lipid accumulation in the RiO using BODIPY (FIG. 6). For quantification, Applicant used ImageJ LUT to convert the BODIPY® intensity in each image from brightness and darkness levels to a numerical value. In addition, Applicant also analyzed iron accumulation in RIO (FIG. S7). It was studied using the ammonium iron sulfate (FAS) treatment and Fe dye FeRhoNox 540/575. Thus, RiO possess multiple human hepatic functions similar to control LO including ALB (albumin) secretion and bile transport function, suggesting the presence of the RFID chip in the liver organoids does not seem to affect native structure and function.

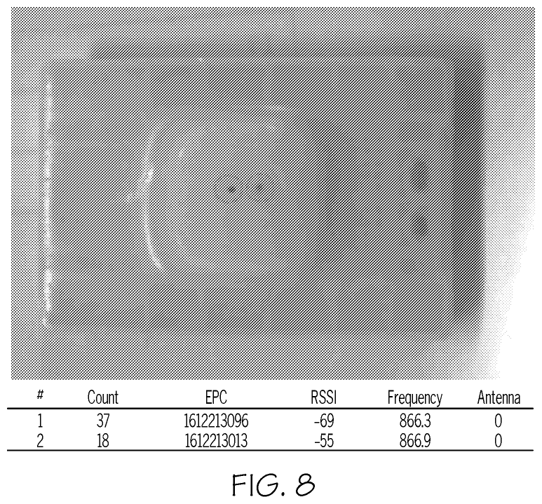

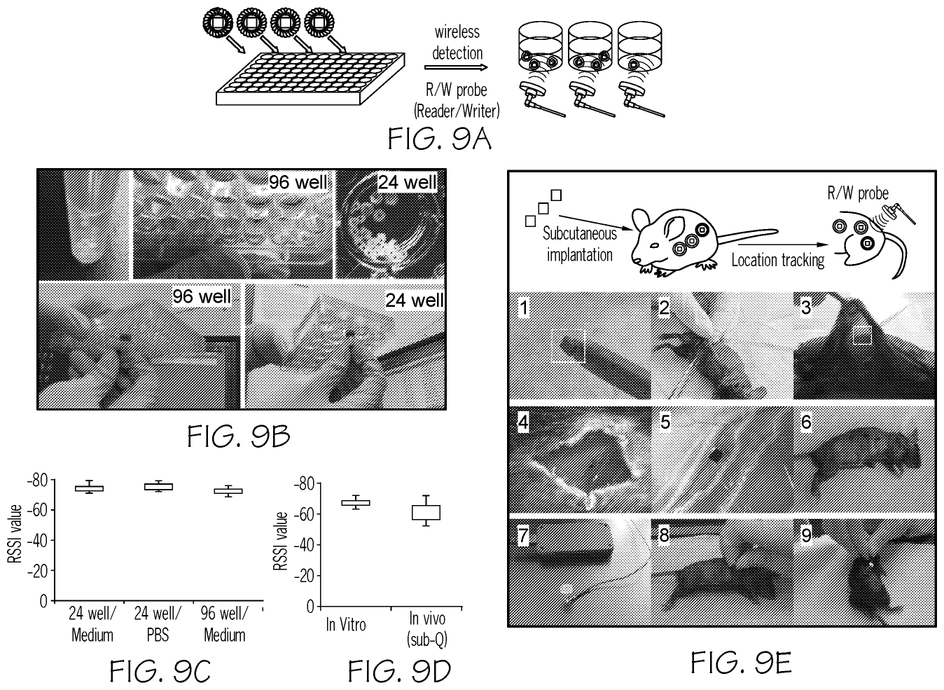

RFID chips are generally highly durable, but their durability in multiple tissue culture contexts has not been examined. To determine their cryopreservation potential under ultralow temperatures, Applicant examined the tolerance of O-Chip to freezing or to submersion in liquid nitrogen. For slow freezing and vitrification, O-Chip may be subjected to low temperatures during storage. A range of temperatures was tested, from 4° C. to −196° C., to determine tolerance and whether efficacy was impacted (Table. 1). In addition, during culture conditions the incorporated O-Chip must also endure dynamic changes in pH. Throughout exposures to these conditions, the RFID tags remained intact and durable, suggesting a high degree of tolerance to low temperatures and pH (Table. 1). Moreover, O-Chip remained functional after routine laboratory sterilization processes such as autoclaving (dry and heat methods). The O-Chip incorporated in paraffin embedded RiO could still be detected wirelessly. Since paraffin embedded process required immersion of O-Chip in water-based solutions and ethanol, these results supported tolerance to O-Chip to solutions and solvents (FIG. 5, FIG. 8). The wireless detection of RiO-derived signals can be possible both in in vitro and in vivo settings (FIG. 9). Collectively, O-Chips functions robustly under a variety of environmental exposures routine to tissue culture protocols.

TABLE 1
Tolerance of RFID

| | | | EPC# | | After processing | | | |
|---|---|---|---|---|---|---|---|---|
| | | # | assignment | | EPC# | RSSI | Frequency | Antenna |
| Freezing | RT | 1 | 1612213035 | → | 1612213035 | −70 | 866.9 | 0 |
| | | 2 | 1612213004 | → | 1612213004 | −70 | 866.9 | 0 |
| | | 3 | 1612213005 | → | 1612213005 | −66 | 866.9 | 0 |
| | | 1 | 1612213081 | → | 1612213081 | −69 | 868.3 | 0 |
| | 4° C. | 2 | 1612213047 | → | 1612213047 | −70 | 865.7 | 0 |
| | | 3 | 1612213087 | → | 1612213087 | −69 | 866.9 | 0 |
| | | 1 | 1612213045 | → | 1612213045 | −68 | 867.5 | 0 |
| | −20° C. | 2 | 1612213080 | → | 1612213080 | −71 | 866.9 | 0 |
| | | 3 | 1612213095 | → | 1612213095 | −69 | 865.7 | 0 |
| | | 1 | 1612213102 | → | 1612213102 | −71 | 866.3 | 0 |
| | −80° C. | 2 | 1612213086 | → | 1612213086 | −70 | 865.7 | 0 |
| | | 3 | 1612213097 | → | 1612213097 | −70 | 865.3 | 0 |
| | −196° C. | 1 | 1612213022 | → | 1612213022 | −69 | 865.7 | 0 |
| Autoclaving | 121° C. | 1 | 1612213026 | → | 1612213026 | −67 | 865.7 | 0 |
| | | 2 | 1612213037 | → | 1612213037 | −69 | 868.9 | 0 |
| | | 3 | 1612213057 | → | 1612213057 | −69 | 865.7 | 0 |
| Acidic | pH 1.0 | 1 | 1612213026 | → | 1612213026 | −67 | 865.7 | 0 |
| environment | | 2 | 1612213037 | → | 1612213037 | −69 | 866.9 | 0 |
| | | 3 | 1612213057 | → | 1612213057 | −69 | 865.7 | 0 |
| | pH 2.0 | 1 | 1612213026 | → | 1612213026 | −67 | 865.7 | 0 |
| | | 2 | 1612213037 | → | 1612213037 | −69 | 866.9 | 0 |
| | | 3 | 1612213057 | → | 1612213057 | −69 | 865.7 | 0 |
| | pH 3.0 | 1 | 1612213026 | → | 1612213026 | −67 | 865.7 | 0 |
| | | 2 | 1612213037 | → | 1612213037 | −69 | 866.9 | 0 |
| | | 3 | 1612213057 | → | 1612213057 | −69 | 865.7 | 0 |
| | pH 4.0 | 1 | 1612213026 | → | 1612213026 | −67 | 865.7 | 0 |
| | | 2 | 1612213037 | → | 1612213037 | −69 | 866.9 | 0 |
| | | 3 | 1612213057 | → | 1612213057 | −69 | 865.7 | 0 |
| | pH 5.0 | 1 | 1612213026 | → | 1612213026 | −67 | 865.7 | 0 |
| | | 2 | 1512213037 | → | 1612213037 | −69 | 866.9 | 0 |
| | | 3 | 1612213057 | → | 1612213057 | −69 | 865.7 | 0 |
| | PH 6.0 | 1 | 1612213026 | → | 1612213026 | −67 | 865.7 | 0 |
| | | 2 | 1612213037 | → | 1612213037 | −69 | 866.9 | 0 |
| | | 3 | 1612213057 | → | 1612213057 | −69 | 865.7 | 0 |

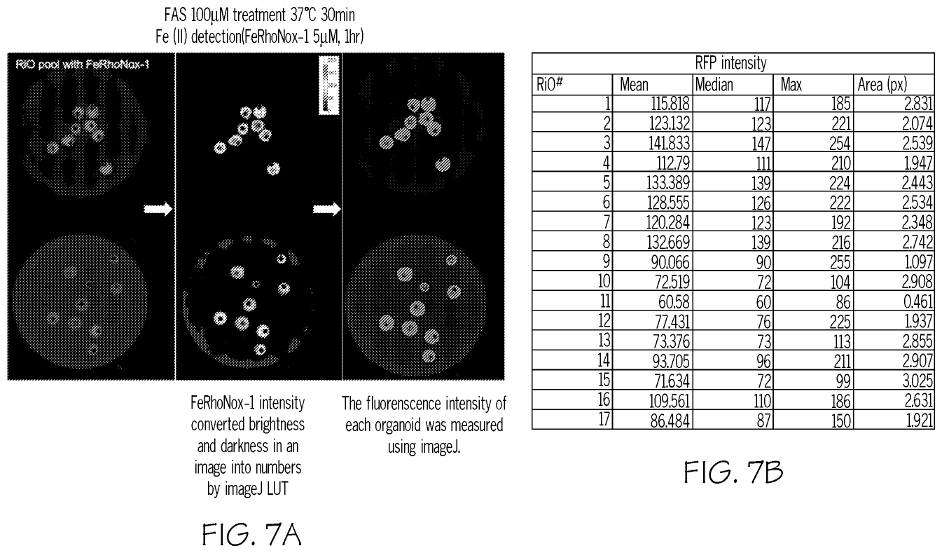

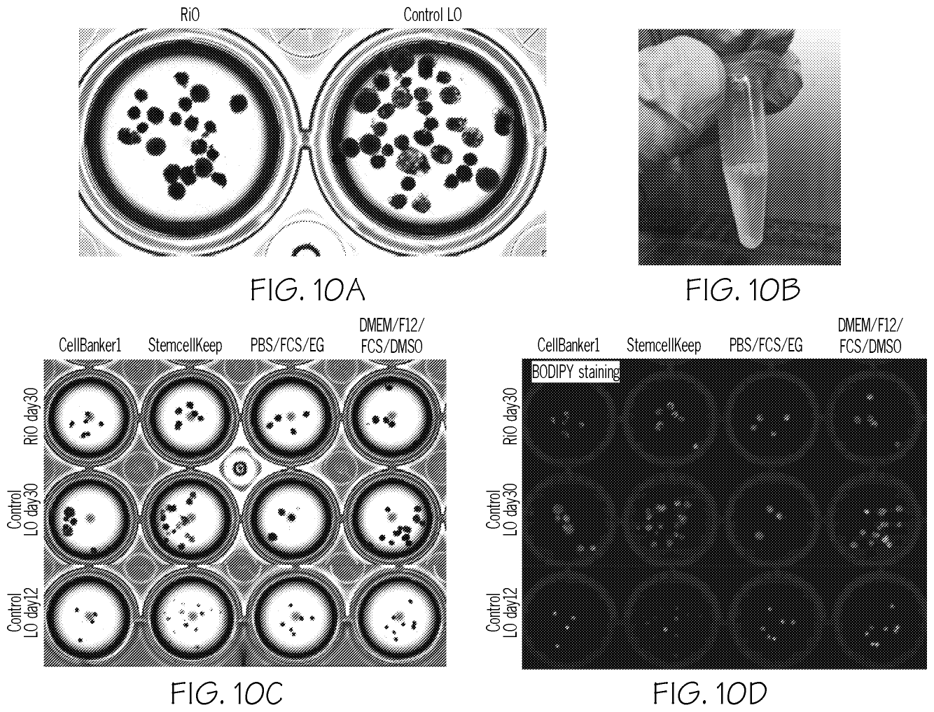

The remarkable durability of O-Chip prompted Applicant to test their cryopreservation potential of RiO. To develop an efficient method for optimal cryopreservation of RiO, Applicant carried out comparative experiments using several different reagents and freezing methods (FIG. 7 A and FIG. 7B). The morphology of RiO was fully preserved after thawing after the gradual freezing method (FIG. 7B), and the O-Chip could be read without being affected. To evaluate the potential for a phenotypic screening assay after freezing and thawing, Applicant examined the previously frozen organoids by inducing steatosis according to unpublished protocols (Ouchi et al., In Revision). The thawed RiO was exposed to free fatty acid, and BODIPY® live-cell staining was performed to detect lipid accumulation. Fluorescence microscopy imaging showed that the cryopreserved RiO had retained the capability for accumulating lipid droplets (FIG. 7D, FIG. 10D). Taken together, cryopreserved RiO not only retained its morphology, but preserved lipid storage.

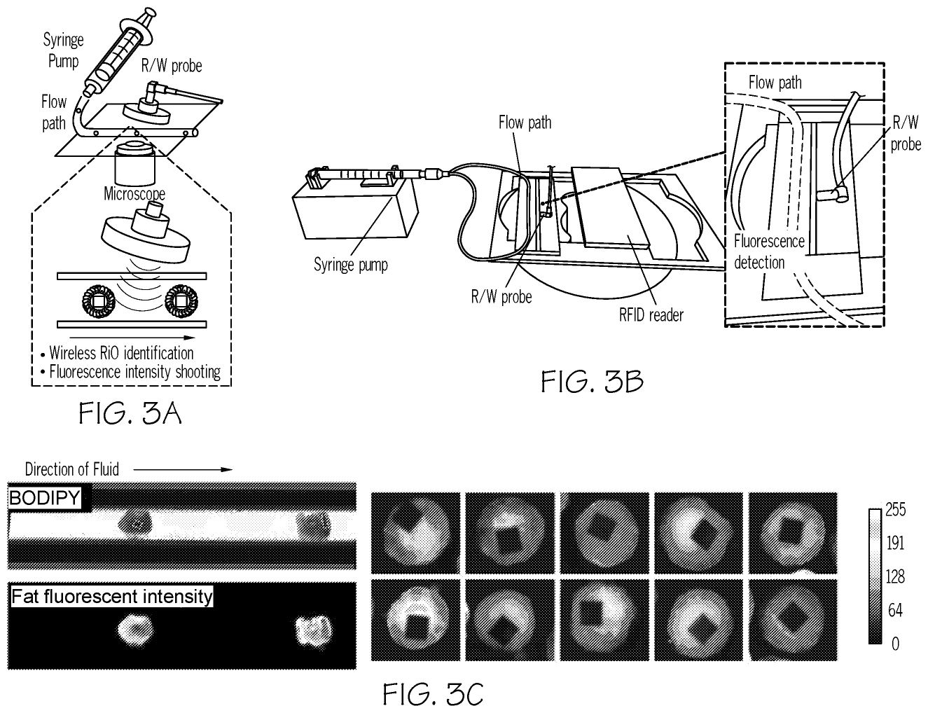

Next, Applicant evaluated how readily RiO can be traced (FIG. 3). Generated RiOs were placed into 96 and 24 well plastic plates (FIG. 3A, 3B). When the excitation RF pulse was applied from the bottom, the O-Chip was detected wirelessly, and Applicant measured their frequencies by measuring Radio Strength Signal Indicator (“RSSI”) values. RSSI indicates the strength of RFID signals and values range from −100 to 0. The closer to 0 RSSI values are, the stronger the signals are. The signals depend on factors such as distance or shielding materials. Comparisons of RSSI measurements of RiOs indicated there were no significant difference amongst the different plate configurations or media supporting the RiOs (FIG. 3C).

Applicant further used the O-Chip system to track the post-transplant location in vivo. To establish the feasibility of this approach, Applicant localized O-Chips into three different locations following subcutaneous implantation in an anesthetized mouse (FIG. 3C). After embedding the O-Chips under mouse skin (FIG. 3D), Applicant measured RSSI values with a reader directly over the mouse's skin. The RSSI of the received signals exhibited three different peaks corresponding to the target locations. A localization accuracy was less than 1,000 μm among measurements for all cases. Compared to the signal strength obtained in a microtube, RSSI of RFID microchips under mouse skin was modestly lower but still detectable (FIG. 3E). Implantation into the abdominal cavity and subcapsule of the kidney did not emit sufficient signals to the receivers and could not be measured. As such, subcutaneous locations may be preferred for optimal RiO tracing in living biological tissues.

To further identify each RiO, Applicant developed a device to simultaneously measure the fluorescence and detect the RFID chip in one step. The measurement workflow and device details for RiO phenotyping are shown in FIG. 3A. Using this detection system, simultaneous Electronic Product Code (EPC) recording and fluorescence imaging of passing RiO are possible through the flow (FIG. 3B). This device was composed of a microliter-scale syringe pump, a flow path through which the organoid passes, a detection probe that recognizes RFID, and a fluorescence microscope. To validate the entire system with RIO, Applicant treated the organoid with fatty acids and then visualized the lipid accumulation in the RiO using a lipid-specific fluorescent dye called BODIPY®. Then, RiOs were passed through the flow path using a syringe pump that can adjust the flow rate on a microscale. Subsequently, the RFID signal was detected together with a snapshot of fluorescence intensity (FIG. 3C). Thus, Applicant successfully developed a higher-throughput detection device for fluorescence imaging based on phenotyping assay coupled with the RFID-integrated organoids.

Understanding the pathological variations in human diseases with human stem cell models is considered by some to be vital to promote precision medicine and drug screening applications. Community efforts to derive a population human induced pluripotent stem cell (iPSC) library from healthy and diseased donors provides an accessible platform to study gene expressional variation such as gene expression quantitative trait loci (eQTL) (Kilpinen et al., 2017)15. However, head-to-head manual comparison among different lines is completely inefficient and unrealistic, and, more importantly, the organoid-level phenotyping method remains to be developed. To circumvent this challenge, Applicant approached the RiO based approach so as to detect the specific donor from pools after identifying a notable phenotype through an initial screen.

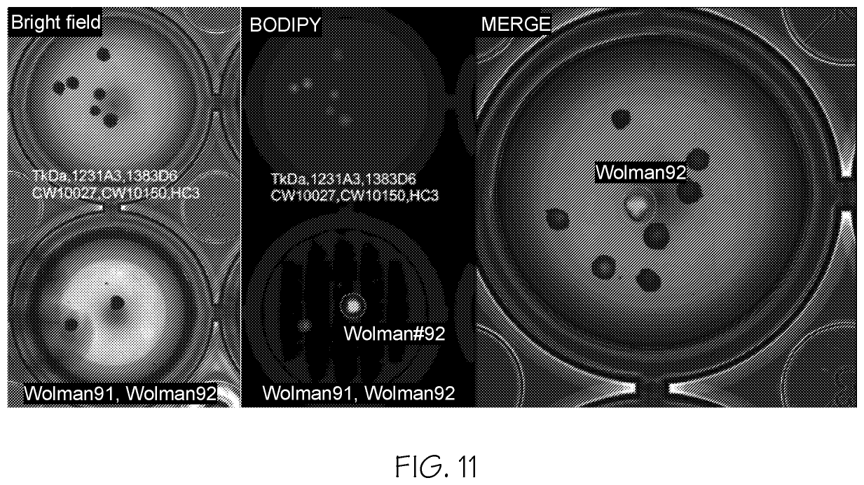

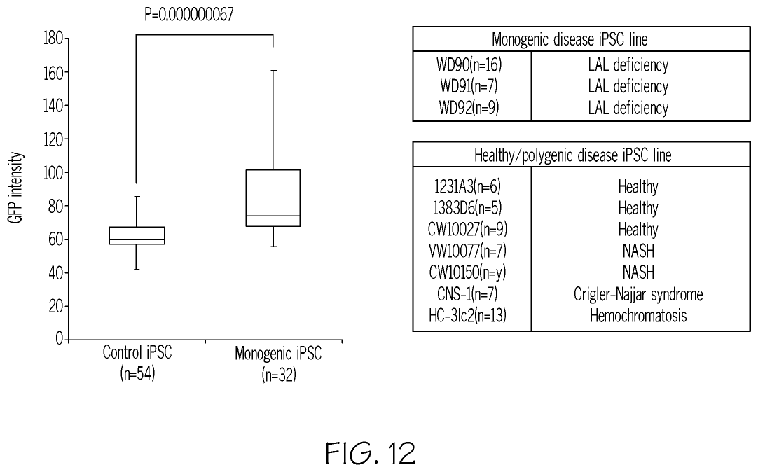

As a proof-of-principle phenotypic screen, Applicant specifically generated a small pool of frozen RiOs (FIG. 4A, FIG. 4B, FIG. 11). Applicant selected seven different donor derived iPSCs that included two patients with a monogenic form of steatohepatitis, called Wolman disease. Wolman disease is caused by the absence of lysosomal acid lipase (LAL), and hepatocytes from Wolman disease patients have heavy lipid accumulation, so measuring lipid accumulation of hepatocytes can provide an effective screen for steatosis. Applicant confirmed prominent steatosis progression in Wolman-derived LO after exposure to free fatty acids (FIG. 8, FIG. 12). Similarly, BODIPY® live-imaging was conducted against the pooled RiO (FIG. 4C). Fluorescent microscopy imaging confirmed significantly higher intensity in specific RiO versus others (FIG. 4D). Applicant identified each RiO donor by its RFID chip and discovered that these higher intensity RiO (indicative of increased steatosis) corresponded to Wolman disease-derived iPSC organoids (FIG. 4D). Thus, these RiO have successfully recapitulated human, genetic-based, steatohepatitis pathology by reflecting human genetic disease. More importantly, a RiO based pooling approach may be an efficient way to determine individualized phenotypes in a high throughput setting.

Net, the Applicant has identified an innovative strategy to track a spectrum of organoid phenotypes by integrating a digital miniaturized RFID into biological tissues, in particular, organoids that maintain their one or more native functions. Applicant has found that, the collective cells experience aggregation via a self-assembling cavitation process, the micro-chips seamlessly locate into the intra-lumen of organoids without compromising native structures, preventing any tissue damage or destruction. Strikingly, the chip-integrated organoids underwent freezing and thawing cycles with no obvious loss of viability, and would, therefore, greatly facilitate the banking efforts of multiple donor derived organoids. Given that the miniaturization of digital devices including biosensors16, robotics17, and cameras18 is rapidly evolving7, 19, 20, the overall strategy exemplified with RFID will enable the development of new integral approaches for the sensing, management and intervention of biological tissues.

About 5,000+ pluripotent stem cell lines have been established and deposited into emerging biobanks such as HipSci, EBiSC, WiCell, Coriell, RIKEN and NYSCF. A recently proposed “cellomics” approach, in which population stem cell array is coupled to the assessment of the phenotypic variabilities of each line, is a highly attractive approach to screen and define the cellular and molecular basis for human variations, such as expression (eQTL)-based association studies (Kilpinen et al., 2017)15. However, conventional laboratory scale protocols are essentially inefficient and not equipped to perform large comparative analyses, as culturing each cell line separately is costly and labor intensive. In contrast, the current RFID market price is around US $0.1-0.2. Even considering that large numbers of stem cell or primary cell lines are used, the overall cost for analysis is by far inexpensive as compared to available cutting-edge single-cell genomics approaches for identifying individual in a pooled condition. Thus, the RFID-based strategy is a cost-effective and therefore scalable approach to enable forward donor identification.

More importantly, organoid-level phenotyping, and not just transcriptomic analysis, is particularly demanding for drug screening or precision medicine applications. RiO-based phenotyping at the organoid level is a different class of approach to determine large scale phenotypes, not just a transcriptomic characterization. By taking advantage of donor identifiable organoids and expanding on existing concepts, a ‘forward cellomics’ approach may be used as a potential strategy to determine the personalized phenotypes in a pooled condition. Forward genetics is a known scalable approach of studying genetic impacts on phenotype as a counter strategy to reverse genetics, which determines the function of a gene by analyzing the phenotypic effects of altered DNA sequences. Likewise, a forward cellomics approach would first determine phenotypic variations by existing screen methods, and then study the causal genetic and cellular bases for pronounced differences. Applicant's data suggests the possibility of this “forward” phenotyping methods, at least, for example, for determining monogenic basis for pathological phenotype of steatohepatitis. By pooling the organoids in the same well, significant cost reduction will be expected, in addition to enhanced reproducibility of the experiments. The disclosed compositions and methods may be coupled to developing high throughput organoid phenotyping assays21, all of which could be extended to other organoid systems or diseases. Nevertheless, the presented methodology is scalable and time and cost-efficient. Digitalized organoids may be used to facilitate the in vitro study of an extensive population cohort by examining variable disease phenotypes, drug safety and efficacy.

### Methods

Maintenance of PSCs. Two human iPSC lines, 1231A3 and 1383D6 were obtained from Kyoto University. The TkDA3-4 line was kindly provided by K. Eto and H. Nakauchi. The CW10027 and CW10150 lines were obtained from the NINDS iPSC Repository at Coriell Institute. The Wolman disease Wolman91 and Wolman92 iPSC lines were obtained from reprogrammed at Cincinnati Children's Hospital Medical Center (CCHMC) Pluripotent Stem Cell Facility co-directed by CN. Mayhew and JM. Wells. Human iPSC lines were maintained as described previously (Takebe et al., 2015; Takebe et al., 2014). Undifferentiated human iPSCs were maintained on feeder-free conditions in mTeSRI medium (StemCell technologies, Vancouver, Canada) on plates coated with Matrigel (Corning Inc., NY, USA) at 1/30 dilution at 37° C. in 5% CO2 with 95% air.

Definitive endoderm induction. Human iPSCs differentiation into definitive endoderm was done using previously described methods with slight modifications 2226. In brief, colonies of human iPSCs were isolated in Accutase (Thermo Fisher Scientific Inc., MA, USA) and 150,000 cells/mL were plated on Matrigel coated tissue culture plates (VWR Scientific Products, West Chester, PA). Medium was changed to RPMI 1640 medium (Life Technologies) containing 100 ng/ml Activin A (R&D Systems, MN, USA) and 50 ng/mL bone morphogenetic protein 4 (BMP4; R&D Systems) on day 1, 100 ng/ml Activin A and 0.2% KnockOut™ Serum Replacement (KSRfetal calf serum (PCS; Thermo Fisher Scientific Inc.) on day 2 and 100 ng/mL Activin A and 2% KSRFCS on day 3. Day 4-6 cells were cultured in Advanced DMEM/F12 (Thermo Fisher Scientific Inc.) with B27 (Life Technologies) and N2 (Gibco, CA, USA) containing 500 ng/ml fibroblast growth factor 4 (FGF4; R&D Systems) and 3 μM CHIR99021 (Stemgent, MA, USA). Cells were maintained at 37° C. in 5% CO2 with 95% air and the medium was replaced every day. Spheroids appeared on the plate at day 7 of differentiation.

RFID chip incorporated liver organoid (RiO) generation. At day 7, definitive endoderm cells were dissociated to single cells in Accutase (Thermo Fisher Scientific Inc., Waltham, MA, USA), spun down and quickly resuspended at the desired concentration in differentiation medium (5.0×105 cells per 100 μL). Differentiation medium was Advanced DMEM/F12 with B27, N2, 2 μM retinoic acid (RA; Sigma, MO, USA) and 10 μM ROCK inhibitor Y-27632 (R&D Systems, Minneapolis, MN). Cells were loaded into 96-well ultra-low attachment (ULA) U bottom plates (Corning, Acton, MA, USA) in 200 μL volume respectively. After seeding the cells, the ultra-compact RFID chip (SK-Electronics Co., Ltd., Japan) was placed in each well and centrifuged for 1 minute at 130 g. Ultra-compact RFID chips are able to purchase from SK-Electronics website (www.skel.co.jp/sales/rfid/en/index). Plates were then incubated overnight at 37° C., 5% CO2. The following day, the medium was replaced with freshly prepared differentiation medium without ROCK inhibitor. The medium was changed every day. After 3 days of culture, the medium was replaced with Hepatocyte Culture Medium (HCM; Lonza, MD, USA) with 10 ng/mL hepatocyte growth factor (HGF; PeproTech, NJ, USA), 0.1 μM Dexamethasone (Dex; Sigma) and 20 ng/mL Oncostatin M (OSM; R&D Systems). After 10-15 days of culture, human iPSC derived RiOs were detached, collected and analyzed.

Albumin ELISA. To measure the albumin secretion level of RIO, RiO were seeded and cultured on 24-well ULA plates (Corning). To define the exact number of RiO in each well, the RiOs were captured on the BZ-X710 Fluorescence Microscope (Keyence, Osaka, Japan). The culture supernatants were collected at a 24 hr time point after the initial culture and stored at −80° C. until use. The supernatant was centrifuged at 1,500 rpm for 3 min to pellet debris, and the resulting supernatant was assayed with the Human Albumin ELISA Quantitation Set (Bethyl Laboratories, Inc., TX, USA) according to the manufacturer's instructions. Significance testing was conducted by Student's t-test.

Whole mount immunofluorescence. RiO were fixed for 30 min in 4% paraformaldehyde and permeabilized for 15 min with 0.5% Nonidet P-40. RiO were washed by 1x PBS three times and incubated with blocking buffer for 1 hr room temperature. RiO were then incubated with primary antibody; anti-albumin antibody (Abeam) and antihepatocyte nuclear factor 4 alpha antibody (Santa Cruz) overnight at 4° C. RiO were washed by 1×PBS and incubated in secondary antibody in blocking buffer for 30 min at room temperature. RiO were washed and mounted using Fluoroshield mounting medium with DAPI (Abeam). The stained RiO were visualized and scanned on a Nikon A1 Inverted Confocal Microscope (Japan) using 60x water immersion objectives.

RNA isolation, RT-qPCR. RNA was isolated using the RNeasy mini kit (Qiagen, Hilden, Germany). Reverse transcription was carried out using the High-Capacity cDNA Reverse Transcription Kit (Thermo Fisher Scientific Inc.) according to manufacturer's protocol. qPCR was carried out using TaqMan gene expression master mix (Applied Biosystems) on a QuantStudio 3 Real-Time PCR System (Thermo Fisher Scientific Inc.). All primers and probe information for each target gene was obtained from the Universal ProbeLibrary Assay Design Center (qpcr.probefinder.com/organism.jsp). Significance testing was conducted by Student's t-test.

Rhodamine123 and Cholyl-Lysyl-Fluorescein transport assay. RiO were incubated with 100 μM of Rhodamine 123 (Sigma) and 5 μM of Cholyl-Lysyl-Fluorescein (CLP, Corning Incorporated) for 10 minutes at 37° C. Next, RiO were washed three times with PBS. Images were captured on the KEYENCE BZ-X710 Fluorescence Microscope (Keyence).

Live-cell imaging of lipid accumulation. RiO from 6 donors were pooled in ULA 24-well plates (Corning) and subjected to live-cell staining. For lipid accumulation, RiOs were treated with 100 μM of oleic acid (Sigma) for 24 hr at 37° C. Quantitative estimation of lipid accumulation was performed by BODIPY® 493/503 (Thermo Fisher Scientific Inc.). Images were captured on the KEYENCE BZ-X710 Fluorescence Microscope (Keyence) and fluorescence intensity was measured by ImageJ 1.48k software (Wayne Rasband, NIHR, USA, imagej.nih.gov/ij).

Cryopreservation and thawing of RiO. For RiO cryopreservation, RiO were washed three times with PBS and resuspended in freezing medium. The following freezing mediums were used: CELLBANKER® 1(AMS Biotechnology Limited, UK), StemCell Keep™ (Abnova), PBS/20%F CS/ethylene glycoll.8M(EG; Sigma) and DMEM/F12(Gibco)/20% FCS/10% dimethyl sulfoxide (DMSO; Sigma). One to five RiOs were suspended in 400 μL of freezing medium and pipetted into a cryovial. CELLBANKER® 1 and StemCell Keep were used according to the manufacturer's protocol. PBS/PCS/EG and DMEM/F12/FCS/DMSO conditions were directly frozen at −80° C. For thawing, frozen RiOs were quickly thawed and washed with culture medium. Thawed RiOs were cultured and analyzed.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### References

1. Finkenzeller, K. Fundamentals and applications in contactless smart cards, radio frequency identification and near-field communication, (Wiley, Chichester, West Sussex; Hoboken, NJ, 2010).
2. Pardal, M. L. & Marques, J. A. Towards the Internet of Things: An Introduction to RFID Technology. Iwrt 2010: Rfid Technology—Concepts, Applications, Challenges, 69-78 (2010).
3. Want, R. An introduction to RFID technology. Ieee Pervas Comput 5, 25-33 (2006).
4. Leung, A.A., et al. Tolerance testing of passive radio frequency identification tags for solvent, temperature, and pressure conditions encountered in an anatomic pathology or biorepository setting. J Pathol Inform 1, 21 (2010).
5. Das, R. RFID Forecasts, Players and Opportunities 2017-2027. (2017).
6. Chai, P. R., et al. Utilizing an Ingestible Biosensor to Assess Real-Time Medication Adherence. J Med Toxicol 11, 439-444 (2015).
7. Chai, P. R., Rosen, R. K. & Boyer, E. W. Ingestible Biosensors for Real-Time Medical Adherence Monitoring: MyTMed. Proc Annu Hawaii Int Conf Syst Sci 2016, 3416-3423 (2016).
8. Chen, L. Y., et al. Mass fabrication and delivery of 3D multilayer muTags into living cells. Sci Rep 3, 2295 (2013).
9. Takebe, T., et al. Vascularized and Complex Organ Buds from Diverse Tissues via Mesenchymal Cell-Driven Condensation. Cell Stem Cell 16, 556-565 (2015).
10. Takebe, T., et al. Vascularized and functional human liver from an iPSC-derived organ bud transplant. Nature 499, 481-484 (2013).
11. Lancaster, M. A. & Knoblich, J. A. Organogenesis in a dish: modeling development and disease using organoid technologies. Science 345, 1247125 (2014).
12. Workman, M. J., et al. Engineered human pluripotent-stem-cell-derived intestinal tissues with a functional enteric nervous system. Nat Med 23, 49-59 (2017).
13. Saini, A. Cystic Fibrosis Patients Benefit from Mini Guts. Cell Stem Cell 19, 425-427 (2016).
14. Burke, P. & Rutherglen, C. Towards a single-chip, implantable RFID system: is a single-cell radio possible? Biomed Microdevices 12, 589-596 (2010).
15. Kilpinen, H., et al. Common genetic variation drives molecular heterogeneity in human iPSCs. Nature 546, 370-375 (2017).
16. Zhong, J., Liu, H. J., Maruyama, H., Masuda, T. & Arai, F. Continuous-wave laser-assisted injection of single magnetic nanobeads into living cells. Sensor Actuat B-Chem 230, 298-305 (2016).
17. Nelson, B. J., Kaliakatsos, I. K. & Abbott, J. J. Microrobots for Minimally Invasive Medicine. Annual Review of Biomedical Engineering, Vol 12 12, 55-85 (2010).
18. Liu, L., Towfighian, S. & Hila, A. A Review of Locomotion Systems for Capsule Endoscopy. IEEE Rev Biomed Eng 8, 138-151 (2015).
19. Bergeles, C. & Yang, G. Z. From passive tool holders to microsurgeons: safer, smaller, smarter surgical robots. IEEE Trans Biomed Eng 61, 1565-1576 (2014).
20. Sitti, M., et al. Biomedical Applications of Untethered Mobile Milli/Microrobots. Proc IEEE Inst Electr Electron Eng 103, 205-224 (2015).
21. Dekkers, J. P., et al. A functional CFTR assay using primary cystic fibrosis intestinal organoids. Nat Med 19, 939-945 (2013).
22. Spence, J. R., et al. Directed differentiation of human pluripotent stem cells into intestinal tissue in vitro. Nature 470, 105-109 (2011).

1. A digitized organoid comprising a detectable sensor, wherein said digitized organoid comprises a lumen, and said detectable sensor is located within said lumen.

2. (canceled)

3. The digitized organoid of claim 1, wherein said detectable sensor is hydrogel-based, electronics-based, printed electronics-based, magnetic-based, micro-robotic based, or a combination thereof.

4. The digitized organoid of claim 1, wherein said detectable sensor is configured to detect and measure one or more parameter selected from pH, temperature, pressure, stiffness, elasticity, viscosity, osmotic pressure, DNA, mRNA, protein, carbohydrate (glucose), oxygen, metabolites (ammonia, urea, lactate,) reactive oxygen species (ROS), or combinations thereof.

5. The digitized organoid of claim 1, wherein said digitized organoid comprises a identifier unique to a donor, wherein said identifier is encoded in said detectable sensor.

6. The digitized organoid of claim 1, wherein said digitized organoid is an organoid selected from a liver organoid, a small intestinal organoid, a large intestinal organoid, a stomach organoid, and combinations thereof.

7. (canceled)

8. (canceled)

9. (canceled)

10. (canceled)

11. (canceled)

12. The digitized organoid of claim 1, wherein said digitized organoid is characterized by having a cavitated structure comprising a polarized structure surrounded by a basement membrane.

13. The digitized organoid of claim 1, wherein said digitized organoid comprises one or more native structures selected from biliary canaliculi, microvilli, and combinations thereof.

14. (canceled)

15. (canceled)

16. (canceled)

17. A method of screening a cell population based on a cell phenotype, comprising the step of detecting at least two features of the organoid of claim 1 in a pooled organoid composition, wherein said first feature is the identity of donor individual, and wherein said second feature is selected from a cell phenotype.

18. The method of claim 17 wherein said cell phenotype is selected from cell viability, pathological cell morphology, cell survival, or combinations thereof.

19. The method of claim 17, wherein said first feature is identified using a detectable sensor located in a lumen of said organoid, and wherein said second feature is identified using a second detection method that does not use the detectable sensor.

20. The method of claim 17, further comprising the step of assaying a donor for a genotype and correlating said genotype with said second feature.

21. The method of claim 17, wherein said second feature is drug toxicity or efficacy, further comprising the step of contacting said pooled organoid composition with a drug of interest.

22. A device for high-throughput, phenotype-based detection of a digitized organoid comprising a detectable sensor, comprising
a. a flow path configured for the flow of said digitized organoid;
b. one or more detection features capable of detecting a signal emitted from said digitized organoid in said flow path, wherein said signal is selected from an RFID recording, a fluorescence image, or combination thereof.

23. The device of claim 22, wherein said digitized organoid is passed through the flow path using a syringe pump having an adjustable flow rate.

24. The device of claim 22, wherein the detectable sensor is a radio frequency identification (RFID) sensor.

25. The digitized organoid of claim 1, wherein the detectable sensor is a radio frequency identification (RFID) sensor.

26. The digitized organoid of claim 1, wherein the organoid is a liver organoid.

27. The digitized organoid of claim 1, wherein the liver organoid is capable of albumin secretion, bile transport, or a combination thereof.

28. The method of claim 19, wherein the detectable sensor is an radio frequency identification (RFID) sensor.

29. The method of claim 19, wherein said second detection method is selected from cell fluorescence, radioactivity, or combinations thereof.
